(19) United States
(12) Patent Application Publication
(10) Pub. No.: US 2008/0069771 A1
(43) Pub. Date: Mar. 20, 2008

(54) Oxygenated polymerized hemoglobin solutions and their uses for tissue visualization

(76) Inventors: Anthony J. Laccetti, North Andover, MA (US); Javed Baqai, Lexington, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/901,462

(22) Filed: Sep. 17, 2007

Related U.S. Application Data
(63) Continuation of application No. PCT/US06/12676, filed on Apr. 5, 2006.
(60) Provisional application No. 60/668,417, filed on Apr. 5, 2005.
(60) Provisional application No. 60/781,400, filed on Mar. 10, 2006.

(51) Int. Cl. C07K 14/805; A61K 49/14; A61K 51/08

(52) U.S. Cl. 424/1.69; 424/9.1; 424/9.34; 530/385

(57) ABSTRACT

An oxygenated Hemoglobin (Hb) solution includes from about 10 g to about 250 g of polymerized Hb per liter of solution. About 80% by weight, or greater, of the polymerized Hb of the oxygenated hemoglobin solution is oxyhemoglobin. About 18% by weight, or less, of the polymerized Hb has a molecular weight of over 500,000 Daltons. About 5% by weight, or less, of the polymerized Hb has a molecular weight equal to or less than 65,000 Daltons. A P₅₀ of the polymerized Hb is in a range of between about 34 and about 46 mm Hg. An endotoxin content of the oxygenated Hb solution is less than about 0.05 endotoxin units per mL. A method of visualizing a tissue or organ of a subject includes the steps of administering to the subject an oxygenated hemoglobin solution as described above, and imaging the tissue, blood vessel or organ with an imaging system. A method of producing an oxygenated Hb solution includes the step of oxygenating a Hb solution that includes polymerized Hb as described above to thereby cause about 80% by weight, or greater, of the polymerized Hb to become oxyhemoglobin.

### RELATED APPLICATIONS

This application is a continuation of International Application No. PCT/US2006/012676, which designated the United States and was filed on Apr. 5, 2006, published in English, which claims the benefit of U.S. Provisional Application No. 60/668,417 filed on Apr. 5, 2005 and U.S. Provisional Application No. 60/781,400 filed on Mar. 10, 2006. The entire teachings of the above-mentioned applications are incorporated herein by reference.

### BACKGROUND OF THE INVENTION

Visualizing tissues and internal organs of the body can allow one to obtain direct visual assessment for better understanding and diagnosis of diseases, such as heart diseases, stroke and cancer. Understanding such diseases at a microscopic level on an in vivo basis can lead to better diagnosis and earlier treatments of the diseases. Various imaging tools have been developed and are available in the art to achieve microscopic images of tissues and critical organs, such as brain, lungs, liver, kidneys, heart, etc. Examples of imaging tools include X-ray, ultrasound, magnetic resonance imaging, infrared (IR) imaging, nuclear imaging and optical coherence tomography systems.

Optical coherence tomography (OCT) generally utilizes near-infrared light to generate tomographic in vivo high resolution images. For example, for imaging coronary arteries of a subject with the OCT, it is necessary to temporarily interrupt the coronary blood flow in order to image the vessel wall because of the scattering effects of flowing red blood cells within the vessel. Typically, flushing the vessel with saline has been used for temporarily interrupting the coronary blood flow. However, flushing the vessel with saline can only be performed for a limited time, typically less than 30 seconds, because of induced ischemia, a condition in which blood flow is restricted to a part of the body of a subject.

Magnetic resonance imaging systems rely on the tendency of atomic nuclei possessing magnetic moments to align their spins with an external magnetic field. For example, visualization of tissue metabolism using a magnetic resonance imaging system can be obtained by imaging H₂O formed during aerobic metabolism. An oxygen-17 (¹⁷O) isotope is relatively stable and suitable for use in magnetic resonance imaging. Magnetic resonance imaging processes using oxygen-17 have utilized ¹⁷O₂ delivered into the body of a subject, for example, via inhalant gases containing ¹⁷O₂ or via perfluorocarbons as oxygen-gas carriers to deliver ¹⁷O₂ to target tissues or organs. However, because of the limited oxygen-carrying capacity of perfluorocarbons and the limited oxygen-absorption into the blood stream of a subject, typically a large volume of such inhalant gases or perfluorocarbons is required for imaging processes. ¹⁷O-labeled oxygen gas is rare and thus expensive. So, a large volume of such imaging agents including ¹⁷O-labeled oxygen gas is not desirable. In addition, large amounts of perfluorocarbons can be hazardous to the subject.

Therefore, there is a need to develop new imaging agents that can overcome or minimize the above-mentioned problems of conventional imaging agents, such as saline and perfluorocarbon oxygen-carriers. In particular, an efficient process of introducing ¹⁷O-labeled oxygen gas into tissues for imaging, for example, with a magnetic imaging system, is needed.

### SUMMARY OF THE INVENTION

It has now been discovered that polymerized hemoglobin solution, such as HEMOPURE® (Biopure, Cambridge, Mass.), can be oxygenated in vitro to convert at least about 80% by weight of the polymerized hemoglobin therein to oxyhemoglobin. It also has now been discovered that such oxygenated polymerized hemoglobin solutions, such as oxygenated HEMOPURE® solutions, can be used for clear visualization of tissues, such as coronary arteries, during OCT imaging, with a relatively low risk of ischemia.

In one embodiment, the invention is directed to an oxygenated hemoglobin solution that includes from about 10 grams to about 250 grams of polymerized hemoglobin per liter of solution. In the oxygenated hemoglobin solution: a) about 80% by weight, or greater, of the polymerized hemoglobin of the oxygenated hemoglobin solution is oxyhemoglobin; b) about 18% by weight, or less, of the polymerized hemoglobin has a molecular weight of over 500,000 Daltons; c) about 5% by weight, or less, of the polymerized hemoglobin has a molecular weight equal to or less than 65,000 Daltons; d) a P₅₀ of the polymerized hemoglobin is in a range of between about 34 and about 46 mm Hg; and e) an endotoxin content of the oxygenated hemoglobin solution is less than about 0.05 endotoxin units per milliliter.

In another embodiment, the invention is directed to a method of visualizing a tissue, blood vessel, or organ of a subject. The method includes the steps of: a) administering to the subject an oxygenated hemoglobin solution as described above; and b) imaging the tissue, blood vessel or organ with an imaging system.

In yet another embodiment, the invention is directed to a method of preparing an oxygenated hemoglobin solution as described above. The method includes the step of oxygenating a hemoglobin solution that includes polymerized hemoglobin, using a filter in a single pass-through to thereby cause about 80% by weight, or greater, of the polymerized hemoglobin to become oxyhemoglobin. In the hemoglobin solution, a) about 18% by weight, or less, of the polymerized hemoglobin has a molecular weight of over 500,000 Daltons; b) about 5% by weight, or less, of the polymerized hemoglobin has a molecular weight equal to or less than 65,000 Daltons; c) a P₅₀ of the polymerized hemoglobin is in a range of between about 34 and about 46 mm Hg; and d) an endotoxin content of the hemoglobin solution is less than about 0.05 endotoxin units per milliliter.

The oxygenated hemoglobin solutions of the invention, such as oxy HEMOPURE® solutions, can provide safe alternatives to conventional imaging agents, such as saline or perfluorocarbon oxygen-carrier. In particular, the oxygenated hemoglobin solutions of the invention can be used to obtain in vivo high resolution images of tissues or internal organs with a relatively low risk of ischemia. In addition, oxygenated hemoglobin solutions of the invention that include ¹⁷O-labeled oxyhemoglobin can be used as physiologically-safe ¹⁷O-labeled-oxygen-gas carriers for visualization of tissues, blood vessels or organs using magnetic resonance imaging systems by imaging H₂¹⁷O formed during the aerobic metabolism. In one embodiment, an oxygenated HEMOPURE® solution was used for clear OCT visualization of coronary arteries without causing ischemia.

### BRIEF DESCRIPTION OF THE DRAWINGS

FIGS. 1A and 1B are schematic diagrams showing one embodiment of an oxygenation system of the invention for preparing an oxygenated hemoglobin solution of the invention.

FIG. 2 is a graph showing absorbance at 1310 nm/depth along a specific tilted diffuse reflector as a depth reference: x, flush with D₂O; *, flush with saline (referenced as H₂O); and ∘, flush with oxy-Hemopure at 80 g/L.

### DETAILED DESCRIPTION OF THE INVENTION

The foregoing will be apparent from the following more particular description of example embodiments of the invention, as illustrated in the accompanying drawings. The drawings are not necessarily to scale, emphasis instead being placed upon illustrating embodiments of the present invention.

The present invention makes it possible for one to access and visualize or image tissues, blood vessels and organs of the body of a subject relatively safely by the use of the oxygenated hemoglobin solutions of the invention. The oxygenated hemoglobin solutions of the invention are generally dispersed into the blood stream of the subject once the solutions are administered to the subject.

Generally, the oxygenated hemoglobin solutions of the invention are prepared in vitro by oxygenating hemoglobin solutions that include polymerized hemoglobin to convert at least about 80%, more preferably at least about 90%, by weight of the polymerized hemoglobin to oxyhemoglobin. In some embodiments, about 18% by weight, or less, of the polymerized hemoglobin that is included in the hemoglobin solutions to be oxygenated has a molecular weight of over 500,000 Daltons; about 5% by weight, or less, of the polymerized hemoglobin that is included in the hemoglobin solutions to be oxygenated has a molecular weight equal to or less than 65,000 Daltons; and an endotoxin content of the hemoglobin solution that is included in the hemoglobin solutions to be oxygenated is less than about 0.5 endotoxin units per milliliter, preferably less than about 0.05 endotoxin units per milliliter. Also, a P₅₀ of the polymerized hemoglobin is in a range of between about 24 and about 46 mm Hg, preferably between about 34 and about 46 mm Hg. The oxygenated hemoglobin solutions of the invention prepared in vitro can also include one or more pharmaceutically acceptable carriers and/or excipients. Examples of such carriers include water, saline solution, dextrose solution and the like. Examples of excipients include sodium chloride and physiologically-acceptable buffers.

The term “P₅₀” is recognized in the art as a term employed to describe the interaction between oxygen gas (O₂) and hemoglobin, and represents the partial pressure of oxygen gas (pO₂) at 50% saturation of hemoglobin. Thus, “a P₅₀ of polymerized hemoglobin” indicates interaction between oxygen gas (O₂) and the polymerized hemoglobin. This interaction is frequently represented as an oxygen dissociation curve with the percent saturation of hemoglobin plotted on the ordinate axis and the partial pressure of oxygen in millimeters of mercury (mm Hg) or torrs plotted on the abcissa. Preferably, a P₅₀ of the polymerized hemoglobin that can be employed in the invention is in a range of between about 24 mm Hg and about 46 mm Hg, more preferably between about 34 mm Hg and about 46 mm Hg.

The term “polymerized,” as used herein, encompasses both inter-molecular and intramolecular polyhemoglobin, with at least 50%, preferably greater than about 95%, of the polymerized hemoglobin of greater than tetrameric form. The polymerized hemoglobin that can be employed for the invention can be prepared by polymerizing or cross-linking with a multifunctional cross-linking agent. Preferably, the polymerized hemoglobin is substantially soluble in aqueous fluids having a pH of 6 to 9 and in physiological fluids.

Suitable examples of cross-linking agents are disclosed in U.S. Pat. No. 4,001,200, the entire teachings of which are incorporated herein by reference. Suitable specific examples of the cross-linking agents include compounds having an aldehyde or dialdehyde functionality, such as formaldehyde, paraformaldehyde, formaldehyde activated ureas such as 1,3-bis(hydroxymethyl)urea, N,N′-di(hydroxymethyl)imidazolidinone prepared from formaldehyde condensation with a urea; compounds bearing a functional isocyanate or isothiocyanate group, such as diphenyl-4,4′-diisothiocyanate-2,2′-disulfonic acid, toluene diisocyanate, toluene-2-isocyanate-4-isothiocyanate, 3-methoxydiphenylmethane-4,4′-diisocyanate, propylene diisocyanate, butylene diisocyanate, and hexamethylene diisocyanate; esters and thioesters activated by strained thiolactones; hydroxysuccinimide esters; halogenated carboxylic acid esters; and imidates. Other examples of the cross-linking agents include derivatives of carboxylic acids and carboxylic acid residues of hemoglobin activated in situ to give a reactive derivative of hemoglobin that will cross-link with the amines of another hemoglobin. Examples of the carboxylic acids include citric, malonic, adipic and succinic acids. Carboxylic acid activators include thionyl chloride, carbodiimides, N-ethyl-5-phenyl-isoxazolium-3′-sulphonate (Woodward's reagent K), N,N′-carbonyldiimidazole, N-t-butyl-5-methylisoxazolium perchlorate (Woodward's reagent L), 1-ethyl-3-dimethyl aminopropylcarbodiimde, and 1-cyclohexyl-3-(2-morpholinoethyl)carbodiimide metho-p-toluene sulfonate. The cross-linking reagent can be a dialdehyde precursor that readily forms a bifunctional dialdehyde in the reaction medium. Suitable dialdehyde precursors include acrolein dimer or 3,4-dihydro-1,2-pyran-2-carboxaldehyde which undergoes ring cleavage in an aqueous environment to give alpha-hydroxy-adipaldehyde. Other precursors, which on hydrolysis yield a cross-linking reagent, include 2-ethoxy-3,4-dihydro-1,2-pyran which gives glutaraldehyde, 2-ethoxy-4-methyl-3,4-dihydro-1,2-pyran which yields 3-methyl glutaraldehyde, 2,5-diethoxy tetrahydrofuran which yields succinic dialdehyde and 1,1,3,3-tetraethoxypropane which yields malonic dialdehyde and formaldehyde from trioxane. Exemplary commercially-available cross-linking reagents include divinyl sulfone, epichlorohydrin, butadiene diepoxide, ethylene glycol diglycidyl ether, glycerol diglycidyl ether, dimethyl suberimidate dihydrochloride, dimethyl malonimidate dihydrochloride, and dimethyl adipimidate dihydrochloride.

Preferred specific examples of the cross-linking agents include glutaraldehyde, succindialdehyde, activated forms of polyoxyethylene and dextran, α-hydroxy aldehydes, such as glycolaldehyde, N-maleimido-6-aminocaproyl-(2′-nitro,4′-sulfonic acid)-phenyl ester, m-maleimidobenzoic acid-N-hydroxysuccinimide ester, succinimidyl 4-(N-maleimidomethyl)cyclohexane-1-carboxylate, sulfosuccinimidyl 4-(N-maleimidomethyl)cyclohexane-1-carboxylate, m-maleimidobenzoyl-N-hydroxysuccinimide ester, m-maleimidobenzoyl-N-hydroxysulfosuccinimide ester, N-succinimidyl(4-iodoacetyl)aminobenzoate, sulfosuccinimidyl(4-iodoacetyl)aminobenzoate, succinimidyl 4-(p-maleimidophenyl)butyrate, sulfosuccinimidyl 4-(p-maleimidophenyl)butyrate, 1-ethyl-3-(3-dimethylaminopropyl)carbodiimide hydrochloride, N,N′-phenylene dimaleimide, and compounds belonging to the bis-imidate class, the acyl diazide class or the aryl dihalide class.

Preferred polymerized hemoglobin that can be employed in the invention includes hemoglobin polymerized by a dialdehyde. As used herein, the “hemoglobin polymerized by a dialdehyde” includes both hemoglobin polymerized by a dialdehyde and hemoglobin polymerized by a dialdehyde precursor that readily forms a bifunctional dialdehyde in the reaction medium. Suitable dialdehyde and dialdehyde precursors are as described above. More preferred polymerized hemoglobin that can be employed in the invention includes hemoglobin polymerized by glutaraldehyde. In some embodiments, the oxygenated hemoglobin solutions of the invention include hemoglobin polymerized by glutaraldehyde, but not pyridoxylated by a pyridoxylating agent, such as pyridoxal 5′ phosphate.

As used herein, the term “endotoxin(s)” means the generally cell-bound lipopolysaccharides produced as a part of the outer layer of gram-negative bacterial cell walls, which under many conditions are toxic. When administered to animals, endotoxins can cause fever, diarrhea, hemorrhagic shock and other tissue damages. By the term “endotoxin unit” (EU) is intended that meaning given by the United States Pharmacopeial Convention of 1983, Page 3014, which defined EU as the activity contained in 0.2 nanograms of the U.S. reference standard lot EC-2. One vial of EC-2 contains 5,000 EU. Preferably, an endotoxin content of the oxygenated hemoglobin solutions of the invention is less than about 0.5 endotoxin units per milliliter, such as less than about 0.25 endotoxin units per milliliter, less than about 0.05 endotoxin units per milliter, or less than about 0.02 endotoxin units per milliter. The endotoxin contents can be measured, for example, by the Limulus Amebocytic Lysate (LAL) assay known in the art.

The oxygenated hemoglobin solutions of the invention preferably have levels of endotoxins, phospholipids, foreign proteins and other contaminants which will not result in a significant immune system response and which are non-toxic to the recipient. Preferably, the oxygenated hemoglobin solutions of the invention are ultrapure. Ultrapure as defined herein, means containing less than 0.05 EU/ml of endotoxin, less than 3.3 nmoles/ml phospholipids and little to no detectable levels of non-hemoglobin proteins, such as serum albumin or antibodies.

Preferably, the polymerized hemoglobin solutions that can be used in the invention include stable polymerized hemoglobin. As used herein, the “stable polymerized hemoglobin” is a hemoglobin-based oxygen carrying composition which does not substantially increase or decrease in molecular weight distribution and/or in methemoglobin content during storage periods at suitable storage temperatures for periods of two years or more, and preferably for periods of two years or more, when stored in a low oxygen environment. Suitable storage temperatures for storage of one year or more are between about 2° C. and about 40° C.

Suitable polymerized hemoglobin solutions that can be used in the invention can be derived from new, old or outdated blood from humans and/or other mammals, such as cattle, bovine, ovine, pigs and sheep. Preferably, the polymerized hemoglobin solutions that can be used in the invention include hemoglobin derived from mammals other than humans. More preferably, the polymerized hemoglobin for the oxygenated hemoglobin solutions of the invention includes hemoglobin derived from bovine.

In a preferred embodiment, the oxygenated hemoglobin solutions of the invention include ¹⁷O-labeled oxyhemoglobin. As used herein, the term “¹⁷O-labeled oxyhemoglobin” includes complexes of hemoglobin and an oxygen gas having ¹⁷O, such as hemoglobin-(¹⁷O)₂, hemglobin-(¹⁷O¹⁶O) and hemglobin-(¹⁷O¹⁸O) complexes, and mixtures thereof. Preferably, at least about 30% by mole of the oxyhemoglobin included in the oxygenated hemoglobin solutions is ¹⁷O-labeled oxyhemoglobin. More preferably, at least about 50%, even more preferably at least about 75%, by mole of the oxyhemoglobin included in the oxygenated hemoglobin solutions is ¹⁷O-labeled oxyhemoglobin.

In a specifically preferred embodiment, at least about 30% by mole of the oxyhemoglobin included in the oxygenated hemoglobin solutions is hemoglobin-(¹⁷O)₂. More preferably, at least about 50%, even more preferably at least about 75%, by mole of the oxyhemoglobin included in the oxygenated hemoglobin solutions is hemoglobin-(¹⁷O)₂.

In another preferred embodiment, the oxygenated hemoglobin solutions of the invention include ¹⁶O-labeled oxyhemoglobin. As used herein, the term “¹⁶O-labeled oxyhemoglobin” includes complexes of hemoglobin and an oxygen gas having ¹⁶O, such as hemoglobin-(¹⁶O)₂, hemglobin-(¹⁶O¹⁷O) and hemglobin-(¹⁶O¹⁸O) complexes, and mixtures thereof, such as a regular oxygen gas.

Oxygen has several isotopes, e.g., oxygen-15 (O¹⁵), oxygen-16 (O¹⁶), oxygen-17 (O¹⁷) and oxygen-18 (O¹⁸). The most common and stable isotope of oxygen is oxygen-16. Oxygen-17 is relatively stable as Oxygen-16, and suitable for use, for example, in magnetic resonance imaging. Oxygen-18 is also relatively stable as Oxygen-16, and suitable for use, for example, in infrared spectroscopic imaging. Oxygen-15 is relatively unstable with a short half life and radioactive. Oxygen molecules (O₂) having any of these oxygen isotopes can be obtained in the art.

In yet another preferred embodiment, the oxygenated hemoglobin solutions of the invention include methemoglobin in an amount of about 15% by weight, or less, more preferably about 5% by weight, or less, based upon the total polymerized hemoglobin of the oxygenated hemoglobin solutions.

The present invention also provides a method of visualizing a tissue or organ of a subject. The method includes the step of administering to the subject an effective imaging amount of an oxygenated hemoglobin solution of the invention as described above. The “effective imaging amount” varies in relation to the imaging targets and the imaging systems desired for the imaging targets. Generally, the effective imaging amount is the dosage range deemed by a technician and other medical staff to be useful in practice. It would be apparent to one skilled in the art how to select a dosage amount in any given situation.

Any suitable imaging systems known in the art can be used for the visualizing methods of the invention. In one embodiment, the imaging system is selected from the group consisting of an optical coherence tomography and a magnetic resonance imaging system, both of which are known in the art, for example in U.S. Pat. Nos. 5,321,501; 5,904,651; 5,592,085 and 5,321,501, the entire teachings of which are incorporated herein by reference.

An optical coherence tomography (OCT) is an imaging technology that utilizes advanced photonics and fiber optics to obtain images and tissue characterization on a microscopic scale. The OCT system typically uses infrared light waves that reflect off the internal microstructure(s) within the biological tissues or organs. The frequencies and bandwidths of infrared light are generally orders of magnitude higher than the conventional medical ultrasound signals, resulting in a greatly increased image resolution. In the OCT system, infrared light is generally delivered to the imaging site(s) through one or more optical fibers sized, for example, about 0.006″ diameter. The imaging guidewire typically contains a complete lens assembly to perform a variety of imaging functions. The guidewire can be deployed independently or integrated into existing therapeutic or imaging catheters. OCT imaging can be performed over approximately the same distance of a biopsy at a high resolution and in real time making the most attractive applications for OCT those where conventional biopsies cannot be performed or are ineffective. Suitable examples of the OCT systems for the invention include OCT systems available from Light Lab™, Westford, Mass. and from Prescient” Medical Inc., Dolylestown, Pa. Visualization of tissues or organs using OCT systems in the invention is generally obtained by flushing a target tissue or organ, such as a coronary artery, with an oxygenated hemoglobin solution of the invention, and then imaging the target tissue or organ with an OCT system.

Magnetic resonance imaging (MRI) systems rely on the tendency of atomic nuclei possessing magnetic moments to align their spins with an external magnetic field. Thus, MRI systems produce images indicative of the magnetic properties of tissues. Visualization of tissues or organs using MRI systems in the invention is generally obtained by imaging water, such as ¹⁷O-labeled water, formed during aerobic metabolism. For example, using a ¹H -NMR magnetic reasonance system, the generation of H₂O¹⁷ as a metabolite can be visualized. Localized metabolic activity under physiological conditions can be visualized by monitoring the in vivo production of H₂O¹⁷ in tissue promoted proton T₂ relaxation enhancement. Existing MRI units can be used for this embodiment, as an example at a field strength of 1.0 Tesla.

In one embodiment, an OCT system is used for the visualization methods of the invention. In this embodiment, an oxygenated hemoglobin solution of the invention that is administered to a subject can include oxyhemoglobin having any type of oxygen molecule, e.g., isotopes of oxygen. Preferably, the oxygenated hemoglobin solution includes ¹⁶O-labeled oxyhemoglobin.

In another embodiment, an MRI system is used for the visualization methods of the invention. In this embodiment, an oxygenated hemoglobin solution of the invention that is administered to a subject includes ¹⁷O-labeled oxyhemoglobin.

Examples of the imaging targets for the visualization methods of the invention include arteries, such as coronary arteries, the brain, the heart, visceral tissues and transplants.

The oxygenated hemoglobin solutions for the visualization methods of the invention can be administered to a subject in any suitable means known in the art, depending upon the imaging targets and the imaging systems. In one embodiment, the oxygenated hemoglobin solutions are administered to a subject by infusion into the subject's blood stream. For example, a medical infusion pump (e.g., syringe design, up to 180 mL volume capacity) is used to infuse product through a surgically implanted catheter of narrow diameter.

The present invention also includes methods of preparing an oxygenated hemoglobin solution of the invention. Any suitable methods known in the art can be used for oxygenating in vitro the hemoglobin solutions described above.

In a preferred embodiment, the hemoglobin solutions are oxygenated in vitro with the use of a filter in a single flow-through, whereby an oxygen gas makes contact with the hemoglobin solution within the hydrophobic pores of the filter, diffuses into the hemoglobin solution therein and binds the polymerized hemoglobin of the hemoglobin solution to produce oxyhemoglobin. As used herein, the term “single flow-through” means that the hemoglobin solution to be oxygenated flows through the filter only once, as opposed to re-circulating the solution through the filter.

Preferably, the filter for the oxygenation methods of the invention is a hydrophobic hollow fiber cartridge. The hydrophobic hollow fiber cartridge refers to a membrane-based oxygenator or gas transfer membrane contactor, known in the art. The hollow fiber cartridges are typically made of hydrophobic polymers, such as polyethylene, polypropylene, or PTFE and are of pore sizes preferably from 0.01 to 0.2 microns. Commercially available hydrophobic hollow fiber membrane contactors include the following: Liqui-Cel mini membrane contactors (G477, Celgard LLC, Division of Membrana, Charlotte, N.C.); FiberFlow hydrophobic capsule filter (SV-C-030-P, Minntech Corporation, Minnetonka, Minn.); and Cell-Pharm Hollow Fiber Oxygenators (Oxy-1, Biovest International, Worcester, Mass.) Technologies. The modules are preferably on the order of 0.5-25 square feet, such as 0.5-5 square feet, 0.5-2.5 square feet or 0.5-1.5 square feet, of membrane area and are composed of materials which can be sterilized by either autoclaving or gamma-irradiation. Preferably, the hemoglobin solution to be oxygenated flows through the hydrophobic hollow fiber cartridge in a different direction than a direction of flow of the oxygen gas, such as in an opposite direction.

The hemoglobin solution to be oxygenated flows through the hydrophobic hollow fiber cartridge at a flow rate preferably in a range of between about 2 mL/minute and about 12 mL/minute, such as between about 4 mL/minute and about 12 mL/minute or between about 10 mL/minute and about 12 mL/minute.

The oxygen gas flows through the hydrophobic hollow fiber cartridge at a flow rate preferably in a range of between about 3 cc/minute and about 25 cc/minute, such as between about 3 cc/minute and about 20 cc/minute or between about 10 cc/minute and about 20 cc/minute.

In a preferred embodiment, when the hemoglobin solution to be oxygenated and an oxygen gas independently flow through a filter, preferably a hydrophobic hollow fiber cartridge, at flow rates as described above, the surface area of the filter is in a range of between about 0.5 ft² and about 25 ft² (or between about 450 cm² and about 2.5 m²), such as between about 0.5 ft² and about 5 ft² (or between about 450 cm² and about 0.5 m²), between about 0.5 ft² and about 2.5 ft² (or between about 450 cm² and about 0.25 m²), between about 0.5 ft² and about 1.5 ft² (or between about 450 cm² and about 1,500 cm²), between about 0.8 ft² and about 1.2 ft² (or between about 700 cm² and about 1,200 cm²), or about 1 ft² (or between about 900 cm² and about 1,000 cm²).

In another preferred embodiment, the hemoglobin solution to be oxygenated flows through a filter, preferably a hydrophobic hollow fiber cartridge, in a single pass-through at an area normalized flow rate in a range of between about 20 mL/min/m² and about 110 mL/min/m² (or between about 2 mL/min/ft² and about 10 mL/min/ft²); and an oxygen gas flows through the filter at an area normalized flow rate in a range of between about 50 cc/min/m₂ and about 300 cc/min/m² (or between about 5 cc/min/ft² and about 25 cc/min/ft²).

In the invention, the feed hemoglobin solution is typically a deoxygenated hemoglobin solution. As used herein, the term “deoxygenated hemoglobin solution” means that in the solution, the content of oxyhemoglobin is less than about 10% by weight based on the total hemoglobin. Preferably, the deoxygenated hemoglobin solutions that include polymerized hemoglobin are oxygenated by the oxygenation methods of the invention to have at least about 80% oxyhemoglobin by weight based on the total hemoglobin, more preferably at least about 90% oxyhemoglobin by weight based on the total hemoglobin. In a specifically preferred embodiment, the deoxygenated hemoglobin solutions that include polymerized hemoglobin are oxygenated at the aforementioned hemoglobin-solution and oxygen-gas flow rates through a hydrophobic hollow fiber cartridge having the aforementioned surface areas, to have at least about 80% oxyhemoglobin by weight based on the total hemoglobin, more preferably at least about 90% oxyhemoglobin by weight based on the total hemoglobin.

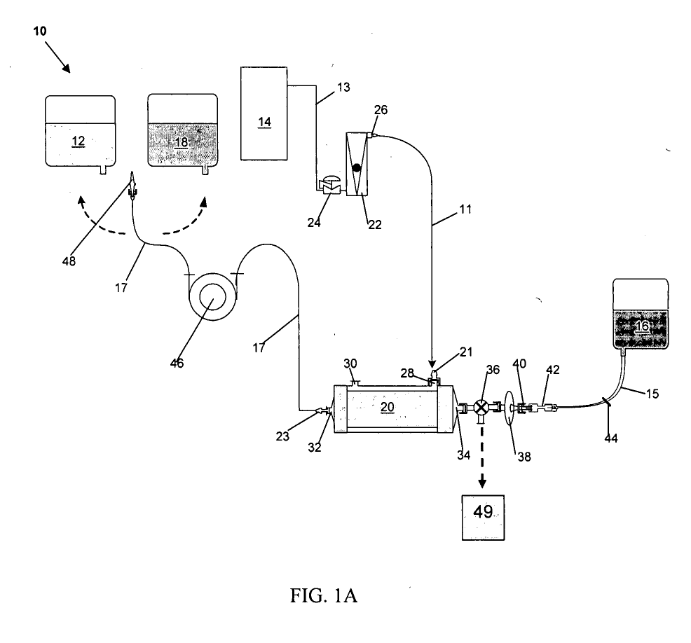

In a specifically preferred embodiment, preparation of an oxygenated hemoglobin solution of the invention is performed using oxygenation system 10 as shown in FIG. 1A. Oxygenation system 10 is a process/apparatus to oxygenate a polymerized hemoglobin solution, such as HEMOPURE® in vitro. A polymerized hemoglobin solution contained in Hb feed bag 12 is pumped through cartridge 20 (preferably, a hydrophobic hollow fiber cartridge) where a gas exchange occurs. Cartridge 20 allows an oxygen gas to diffuse into the polymerized hemoglobin solution and to bind hemoglobin molecules of the polymerized hemoglobin solution. Preferably, cartridge 20 prevents the polymerized hemoglobin solution from leaking into the gas side of cartridge 20. Typically, cartridge 20 has relatively small-sized pores that can prevent any particles and other contaminations from entering to the polymerized hemoglobin solution through gas inlet 28. The resulting oxygenated hemoglobin solutions, such as oxygenated HEMOPURE® solutions, are collected into pre-sterilized, product collection bag 16 via aseptic connections, such as valve 36, connectors 40 and 42 and filter 38. Depending upon the desired uses, the oxygenated hemoglobin solutions are optionally diluted with USP grade saline supplied from saline supply bag 18 to achieve the desired concentration, such as a concentration where the total amount of hemoglobin is in a range of between about 1.0 and about 25 g/dL, such as between about 1.0 and about 17 g/dL, between about 1.0 and about 14 g/dL or between about 1.2 and about 14 g/dL (e.g., about 6.5-13 g/dL). Oxygen supply to cartridge 20 is controlled by connecting pressurized oxygen gas source 14 (e.g., a bottled medical grade oxygen gas or house oxygen supply) to gas inlet 28 of cartridge 20 through medical grade tubings 11 and 13. An oxygen-gas supply pressure is controlled by pressure regulator 24, and a gas flow is controlled by rotameter 22.

In oxygenation system 10, an oxygen gas enters from oxygen gas source 14 to gas inlet 28 of cartridge 20, contacts hollow fibers of cartridge 20 in an opposite direction to the hemoglobin flow and vents to atmosphere or to a gas collection bag (not shown) through gas outlet port 30 of cartridge 20.

Oxygenation system 10 can allow multiple polymerized hemoglobin solutions to be oxygenated and collected continuously in pre-sterilized product collection bags 16.

In some embodiments, oxygenation system 10 is portable. Optionally, once all of the desired numbers of the oxygenated hemoglobin solutions of the invention have been prepared, the connectors, cartridge and associated tubings are discarded.

In a specifically preferred embodiment, all of the materials necessary for oxygenation system 10, such as tubings, fittings, valves, connectors, cartridge and filters, are sterilized prior to use either by autoclaving at an elevated temperature, such as about 121° C., or by gamma irradiation.

Preferably, oxygen gas source 14, such as a medical grade bottle or facility supply, regulated to a supply pressure of less than about 300 psig, more preferably less than about 100 psig, is attached to the inlet of pressure regulator 24 through tubing 13. Preferably, pressure regulator 24 is adjusted for a feed pressure of between about 5 psig and about 10 psig.

Detailed exemplary procedures for setting up and operating oxygenation system 10 are described below:

A. Preparation of Oxygenation System Components

For assembly 50 shown in FIG. 1B, a pre-assembled, gamma-irradiated assembly can be used. Gamma-irradiation is well known in the art and may be performed by a medical and bioprocess product vendor in the art, such as Charter Medical Ltd, Winston-Salem, N.C. Alternatively, non-sterile components are assembled and steam sterilized in a validated autoclave known in the art. Generally, the autoclaved components should be used within seven days of autoclaving. An exemplary procedure for preparing autoclaved assembly 50 is as follows:

a. Pump 46 (e.g., a peristaltic pump) and tubing 17 are installed as shown in FIG. 1A. For valve 36, a 3-way stopcock is demonstrated herein.

b. waste collection bag 49 is attached to 3-way stopcock 36 via a waste line, and 3-way stopcock 36 is directed to waste collection bag 49.

c. Between about 600 and about 800 mL of USP purified water is placed in a clean depyrogenated glass flask.

d. Tubing 17 is submerged in the USP purified water of the glass flask, and the USP purified water is pumped from the glass flask through assembly 50 and into waste collection bag 49 by operating pump 46 at or greater than about 100 mL/min.

e. After flushing with USP purified water, pump 46 is stopped, the waste line is removed from 3-way stopcock 36, and connector 40 (e.g., a female by female Luer connector) is then connected to 3-way stopcock 36.

f. Assembly 50, including tubing 17, cartridge 20, valve 36, filter 38, connectors 23, 40 and 42, are placed in an autoclave pouch.

g. An autoclave pouch containing assembly 50 is placed in a validated autoclave and autoclaved for about 30-40 minutes.

B. Oxygenation System 10 Set Up

The process equipment of oxygenation system 10 can be portable and transported to any designated sites. Preferably, the process equipment is set up at a study site in a clean area where aseptic connections can be made.

Oxygen gas source 14 of a medical grade oxygen gas, regulated to a supply pressure of less than about 300 psig, preferably less than about 100 psig, is attached to pressure regulator 24 and rotameter 22. Pressure regulator 24 is adjusted for a feed pressure of about 5-10 psig. Flexible medical grade tubing 11 is connected from the outlet of rotameter 22 to cartridge 20 through connectors 21 and 26, such as barbed Luer fittings.

Product collection bag 16 (e.g., 1000 mL) is connected to aseptic connector 42.

Hb Feed bag 12 containing a polymerized hemoglobin solution (e.g., HEMOPURE®) or saline supply bag 18 containing USP grade saline is connected to assembly 50 via supply port 48 (e.g., a spike port), for example, by puncturing a spike port of Hb feed bag 12 or saline supply bag 18 with spike port 48.

Prior to the preparation of oxygenated hemoglobin solutions, optionally the assembled components are flushed with saline. Saline is used to prime oxygenation system 10 and can be only required prior to oxygenating the very first bag. One bag of medical (USP) grade saline (e.g., 250 ml) is attached to supply port 48 (e.g., a spike port); one empty waste collection bag 49 (e.g., 1000 ml) is attached to 3-way stopcock 36; and 3-way stopcock 36 is directed towards the attached waste collection bag 49. The pump speed is set at approximately 250 rpm (approximately 75 ml/min) and the entire contents of the saline supply bag are flushed through assembly 50 and collected into the attached waste collection bag 49. Once the saline supply bag has emptied, the pump is stopped and the waste bag is removed and discarded. Subsequently, 3-way stopcock 36 is directed toward product collection bag 16. Oxygenation system 10 is now primed with saline and ready to produce oxygenated hemoglobin solutions, such as oxygenated HEMOPURE® solutions.

C. Oxygen Flow Procedure

Oxygen gas source 14 has an appropriate pressure (e.g., 10-300 psig, preferably 10-100 psig). A suitable pressure rated hose/tubing is provided for tubings 13 and 11. Oxygen gas source 14 is connected via tubing 13 to gas pressure regulator 24. Tubing 13 and gas pressure regulator 24 are connected with each other by appropriate connectors (e.g., metric or English compression connections). Gas pressure regulator 24 is then adjusted to provide a desired pressure, such as about 5-10 psig of oxygen pressure, to rotameter 22. Subsequently, the rotameter's metering valve is adjusted so that the meter's ball is set at a desired range, for example a between about 10 cc/min and 20 cc/min range. The gas flow setting preferably is checked periodically, e.g., the beginning, during and the end of oxygenation processes.

D. Polymerized Hemoglobin Oxygenation

The empty saline supply bag used for flushing assembly 50 is removed from supply port 48, and Hb supply bag 12 is connected to supply port 48. Product collection bag 16 is attached to connector 42 through tubing 15, and clamp 44 is opened. Pump 46 is then started, the polymerized hemoglobin of Hb supply bag 12 is oxygenated within cartridge 20, and the resulting oxygenated hemoglobin solution is collected in product collection bag 16.

E. Saline Dilution

For an optional saline dilution, empty Hb supply bag 12 is removed from supply port 48, and saline supply bag 18 is then connected to supply port 48. Pump 46 is turned on and saline from saline supply bag 18 is transferred to product collection bag 16, diluting the oxygenated hemoglobin solution therein. Once saline supply bag 18 is emptied or once the desired amount of saline is supplied, pump 46 is stopped. Tubing 15 is then clamped and product collection bag 16 is detached from connector 42. The detached product collection bag 16 is then labeled with an approved label and placed on ice or in a refrigerator. Cooling generally maintains a low methemoglobin concentration following the filling at room temperature.

Preferably, the oxygenated hemoglobin solutions of the invention are stored at a temperature of about 15° C. or less. More preferably, the temperature is maintained in a range between about 2° C and about 8° C.

Although oxygenation system 10 is illustrated herein to employ one cartridge 20, in some embodiments, more than one cartridge 20 in series or in parallel can be employed. When a plurality of cartridges 20 is employed in parallel, more than one product collecting bag 16 can be employed and connected to each cartridge. More than one oxygen gas source 14 can also be used in these embodiments.

Polymerized hemoglobin that can be used in preparing the oxyhemoglobin solutions of the invention can be prepared by procedures known in the art, including red blood cell (RBC) collection, purification of the RBC, hemoglobin polymerization and purification of the polymerized hemoglobin. Typically, during the procedures, the blood solution, RBCs and hemoglobin are maintained under conditions sufficient to minimize microbial growth, or bioburden, such as maintaining temperature at less than about 20° C. and above 0° C. Detailed descriptions about the preparation and purification of polymerized hemoglobin (Hb) solutions suitable for the invention can be found in U.S. Pat. Nos. 5,084,558; 5,955,581; 5,753,616; 5,854,209; 5,691,453; 5,691,452; 5,808,011; 5,952,470; 5,895,810; and 5,840,852, the entire teachings of which are incorporated herein by reference.

Suitable RBC sources include human blood, bovine blood, ovine blood, porcine blood, blood from other vertebrates and transgenically-produced hemoglobin, such as the transgenic Hb described in BIOTECHNOLOGY, 12: 55-59 (1994).

The blood can be collected from live or freshly slaughtered donors. One method for collecting bovine whole blood is described in U.S. Pat. Nos. 5,084,558 and 5,296,465, the entire teachings of which are incorporated herein by reference.

In one example, at or soon after collection, the blood is mixed with at least one anticoagulant to prevent significant clotting of the blood. Suitable anticoagulants for blood are as classically known in the art and include, for example, sodium citrate, ethylenediaminetetraacetic acid and heparin. When mixed with blood, the anticoagulant may be in a solid form, such as a powder, or in an aqueous solution.

The blood solution source can be from a freshly collected sample or from an old sample, such as expired human blood from a blood bank. Further, the blood solution could previously have been maintained in frozen and/or liquid state.

Optionally, prior to introducing the blood solution to anticoagulants, antibiotic levels in the blood solution, such as penicillin, are assayed. Antibiotic levels are determined to provide a degree of assurance that the blood sample is not burdened with an infecting organism by verifying that the donor of the blood sample was not being treated with an antibiotic. Examples of suitable assays for antibiotics include a penicillin assay kit (Difco, Detroit, Mich.) employing a method entitled “Rapid Detection of Penicillin in Milk”. It is preferred that blood solutions contain a penicillin level of less than or equal to about 0.008 units/ml. Alternatively, a herd management program to monitor the lack of disease in or antibiotic treatment of the cattle may be used.

Preferably, the blood solution is strained prior to or during the anticoagulation step, for example by straining, to remove large aggregates and particles. A 600 mesh screen is an example of a suitable strainer.

The RBCs in the blood solution are then washed by suitable means, such as by diafiltration or by a combination of discrete dilution and concentration steps with at least one solution, such as an isotonic solution, to separate RBCs from extracellular plasma proteins, such as serum albumins or antibodies (e.g., immunoglobulins (IgG)). It is understood that the RBCs can be washed in a batch or continuous feed mode.

Acceptable isotonic solutions are as known in the art and include solutions, such as a citrate/saline solution, having a pH and osmolarity which does not rupture the cell membranes of RBCs and which displaces the plasma portion of the whole blood. A preferred isotonic solution has a neutral pH and an osmolarity between about 285-315 mOsm. A preferred isotonic solution is composed of an aqueous solution of sodium citrate dihydrate (6.0 g/l) and of sodium chloride (8.0 g/l).

Water which can be used in the method of invention includes distilled water, deionized water, water-for-injection (WFI) and/or low pyrogen water (LPW). WFI, which is preferred, is deionized, distilled water that meets U.S. Pharmacological Specifications for water-for-injection. WFI is further described in Pharmaceutical Engineering, 11, 15-23 (1991). LPW, which is preferred, is deionized water containing less than 0.002 EU/ml.

The isotonic solution can be filtered prior to being added to the blood solution. Examples of suitable filters include a Millipore 10,000 Dalton ultrafiltration membrane, such as a Millipore Cat # CDUF 050 G1 filter or A/G Technology hollow fiber, 10,000 Dalton (Cat # UFP-10-C-85).

RBCs in the blood solution can be washed by diafiltration. Suitable diafilters include microporous membranes with pore sizes which will separate RBCs from substantially smaller blood solution components, such as a 0.1 μm to 0.5 μm filter (e.g., a 0.2 μm hollow fiber filter, Microgon Krosflo II microfiltration cartridge). Concurrently, a filtered isotonic solution is added continuously (or in batches) as makeup at a rate equal to the rate (or volume) of filtrate lost across the diafilter. During RBC washing, components of the blood solution which are significantly smaller in diameter than RBCs, or are fluids such as plasma, pass through the walls of the diafilter in the filtrate. RBCs, platelets and larger bodies of the diluted blood solution, such as white blood cells, are retained and mixed with isotonic solution, which is added continuously or batchwise to form a dialyzed blood solution.

Alternatively, the RBCs can be washed through a series of sequential (or reverse sequential) dilution and concentration steps, wherein the blood solution is diluted by adding at least one isotonic solution, and is concentrated by flowing across a filter, thereby forming a dialyzed blood solution.

RBC washing is complete when the level of plasma proteins contaminating the RBCs has been substantially reduced (typically at least about 90%). Typically, RBC washing is complete when the volume of filtrate drained from diafilter 34 equals about 300%, or more, of the volume of blood solution contained in the diafiltration tank prior to diluting the blood solution with filtered isotonic solution. Additional RBC washing may further separate extracellular plasma proteins from the RBCs. For instance, diafiltration with 6 volumes of isotonic solution may remove at least about 99% of IgG from the blood solution.

The dialyzed blood solution is then exposed to means for separating the RBCs in the dialyzed blood solution from the white blood cells and platelets, such as by centrifugation.

It is understood that other methods generally known in the art for separating RBCs from other blood components can also be employed. For example, sedimentation, wherein the separation method does not rupture the cell membranes of a significant amount of the RBCs, such as less than about 30% of the RBCs, prior to RBC separation from the other blood components.

Following separation of the RBCs, the RBCs are lysed by a means for lysing RBCs to release hemoglobin from the RBCs to form a hemoglobin-containing solution. Lysis means can use various lysis methods, such as mechanical lysis, chemical lysis, hypotonic lysing or other known lysing methods which release hemoglobin without significantly damaging the ability of the Hb to transport and release oxygen.

When recombinantly produced hemoglobin is used, the bacteria cells containing the hemoglobin are washed and separated from contaminants as described above. These bacteria cells are then mechanically ruptured by means known in the art, such as a ball mill, to release hemoglobin from the cells and to form a lysed cell phase. This lysed cell phase is then processed as is the lysed RBC phase.

Following lysis, the lysed RBC phase is then ultrafiltered to remove larger cell debris, such as proteins with a molecular weight above about 100,000 Daltons. Generally, cell debris include all whole and fragmented cellular components with the exception of Hb, smaller cell proteins, electrolytes, coenzymes and organic metabolic intermediates. Acceptable ultrafilters include, for example, 100,000 Dalton filters made by Millipore (Cat # CDUF 050 H1) and made by A/G Technology (Needham, Mass.; Model No. UFP100E55).

The concentrated Hb solution can then be directed into one or more parallel chromatographic columns to further separate the hemoglobin by high performance liquid chromatography from other contaminants such as antibodies, endotoxins, phospholipids and enzymes and viruses. Examples of suitable media include anion exchange media, cation exchange media, hydrophobic interaction media and affinity media. Specific examples of the suitable media include an anion exchange medium suitable to separate Hb from non-hemoglobin proteins. Suitable anion exchange mediums include, for example, silica, alumina, titania gel, cross-linked dextran, agarose or a derivatized moiety, such as a polyacrylamide, a polyhydroxyethyl-methacrylate or a styrene divinylbenzene, that has been derivatized with a cationic chemical functionality, such as a diethylaminoethyl or quaternary aminoethyl group. A suitable anion exchange medium and corresponding eluants for the selective absorption and desorption of Hb as compared to other proteins and contaminants, which are likely to be in a lysed RBC phase, are readily determinable by one of reasonable skill in the art.

Optionally, a method can be used to form an anion exchange media from silica gel, which is hydrothermally treated to increase the pore size, exposed to γ-glycidoxy propylsilane to form active epoxide groups and then exposed to C₃H₇(CH₃)NCl to form a quaternary ammonium anion exchange medium. This method is described in the Journal of Chromatography, 120:321-333 (1976), which is incorporated herein by reference in its entirety.

In one specific example, chromatographic columns are first pre-treated by flushing with a first eluant which facilitates Hb binding. Concentrated Hb solution is then injected onto the medium in the columns. After injecting the concentrated Hb solution, the chromatographic columns are then successively washed with different eluants to produce a separate, purified Hb eluate.

Generally, a pH gradient is used in chromatographic columns to separate protein contaminants, such as the enzyme carbonic anhydrase, phospholipids, antibodies and endotoxins from the Hb. Each of a series of buffers having different pH values, are sequentially directed to create a pH gradient within the medium in the chromatographic column. The use of pH gradients to separate Hb form non-hemoglobin contaminants is further described in U.S. Pat. No. 5,691,452, filed Jun. 7, 1995, which are incorporated herein by reference.

An example of the first buffer is a tris-hydroxymethyl aminomethane (Tris) solution (concentration about 20 mM; pH about 8.4 to about 9.4). An example of the second buffer is a mixture of the first buffer and a third buffer, with the second buffer having a pH of about 8.2 to about 8.6. An example of the third buffer is a Tris solution (concentration about 50 mM; pH about 6.5 to about 7.5). An example of the fourth buffer is a NaCl/Tris solution (concentrations about 1.0 M NaCl and about 20 mM Tris; pH about 8.4 to about 9.4, preferably about 8.9-9.1).

Typically, the buffers used are at a temperature between about 0° C. and about 50° C. Preferably, buffer temperature is about 12.4±1.0° C. during use. In addition, the buffers are typically stored at a temperature of about 9° C. to about 11° C.

The Hb eluate is then preferably deoxygenated prior to polymerization to form a deoxygenated Hb solution by means that substantially deoxygenate the Hb without significantly reducing the ability of the Hb in the Hb eluate to transport and release oxygen, such as would occur from denaturation of formation of oxidized hemoglobin (metHb).

The deoxygenated-Hb is then preferably equilibrated with a low oxygen content storage buffer, containing a sulfhydryl compound, to form an oxidation-stabilized deoxygenated Hb. Suitable sulfhlydryl compounds include non-toxic reducing agents, such as N-acetyl-L-cysteine (NAC) D,L-cysteine, γ-glutamyl-cysteine, glutathione, 2,3-dimercapto-1-propanol, 1,4-butanedithiol, thioglycolate, and other biologically compatible sulfhydryl compounds. The oxygen content of a low oxygen content storage buffer must be low enough not to significantly reduce the concentration of sulfhydryl compound in the buffer and to limit oxyhemoglobin content in oxidation stabilized deoxygenated Hb to about 20% or less, preferably less than about 10%. Typically, the storage buffer has a pO₂ of less than about 50 torr.

The amount of a sulfhydryl compound mixed with the deoxygenated Hb is an amount high enough to increase intramolecular cross-linking of Hb during polymerization and low enough not to significantly decrease intermolecular cross-linking of Hb molecules, due to a high ionic strength. Typically, about one mole of sulfhydryl functional groups (—SH) are needed to oxidation stabilize between about 0.25 moles to about 5 moles of deoxygenated Hb.

Optionally, prior to transferring the oxidation-stabilized deoxygenated Hb to a polymerization reactor, an appropriate amount of water is added to the polymerization reactor.

The pO₂ of the water in the polymerization step is generally reduced to a level sufficient to limit HbO₂ content to about 20%, typically less than about 50 torr. And then the polymerization reactor is blanketed with an inert gas, such as nitrogen. The oxidation-stabilized deoxygenated Hb is then transferred into the polymerization reactor, which is concurrently blanketed with an appropriate flow of an inert gas.

The temperature of the oxidation-stabilized deoxygenated Hb solution in polymerization reactor is raised to a temperature to optimize polymerization of the oxidation-stabilized deoxygenated Hb when contacted with a cross-linking agent. Typically, the temperature of the oxidation-stabilized deoxygenated Hb is about 25EC to about 45EC, and preferably about 41EC to about 43EC throughout polymerization.

The oxidation-stabilized deoxygenated Hb is then exposed to a suitable cross-linking agent at a temperature sufficient to polymerize the oxidation-stabilized deoxygenated Hb to form a solution of polymerized hemoglobin (poly(Hb)) over a period of about 2 hours to about 6 hours.

Examples of suitable cross-linking agents are as described above. In a specific example, glutaraldehyde is used as the cross-linking agent. Typically, about 10 to about 70 grams of glutaraldehyde are used per kilogram of oxidation-stabilized deoxygenated Hb. In a more specific example, glutaraldehyde is added over a period of five hours until approximately 29-31 grams of glutaraldehyde are added for each kilogram of oxidation-stabilized deoxygenated Hb.

A suitable amount of a cross-linking agent is that amount which will permit intramolecular cross-linking to stabilize the Hb and also intermolecular cross-linking to form polymers of Hb, to thereby increase intravascular retention. Typically, a suitable amount of a cross-linking agent is that amount wherein the molar ratio of cross-linking agent to Hb is in excess of about 2:1. Preferably, the molar ratio of cross-linking agent to Hb is between about 20:1 to 40:1.

In a specific example, the polymerization is performed in a buffer with a pH between about 7.6 to about 7.9, having a chloride concentration less than or equal to about 35 mmolar.

Poly(Hb) generally has significant intramolecular cross-linking if a substantial portion (e.g., at least about 50%) of the Hb molecules are chemically bound in the poly(Hb), and only a small amount, such as less than about 10% are contained within high molecular weight polymerized hemoglobin chains. High molecular weight poly(Hb) molecules are molecules, for example, with a molecular weight above about 500,000 Daltons.

After polymerization, the temperature of the poly(Hb) solution in polymerization reactor is typically reduced to about 15° C. to about 25° C.

Wherein the cross-linking agent used is not an aldehyde, the poly(Hb) formed is generally a stable poly(Hb). Wherein the cross-linking agent used is an aldehyde, the poly(Hb) formed is generally not stable until mixed with a suitable reducing agent to reduce less stable bonds in the poly(Hb) to form more stable bonds. Examples of suitable reducing agents include sodium borohydride, sodium cyanoborohydride, sodium dithionite, trimethylamine, t-butylamine, morpholine borane and pyridine borane. Prior to adding the reducing agent, the poly(Hb) solution is optionally concentrated by ultrafiltration until the concentration of the poly(Hb) solution is increased to between about 75 and about 85 g/l. Suitable ultrafilters are of cartridge construction designed for multiple reuse, rated at 30,000 kilodalton (kD) and contain regenerated cellulose supported membrane (e.g., Millipore Helicon, Cat # CDUF050LT and Amicon, Cat #540430).

The pH of the poly(Hb) solution is then adjusted to the alkaline pH range to preserve the reducing agent and to prevent hydrogen gas formation, which can denature Hb during the subsequent reduction. In one embodiment, the pH is adjusted to greater than 10. The pH can be adjusted by adding a buffer solution to the poly(Hb) solution during or after polymerization. The poly(Hb) is typically purified to remove non-polymerized (i.e. low molecular weight hemoglobin having less than about 65 kD) hemoglobin from higher molecular weight polymerized hemoglobin. This fractionation can be accomplished by dialfiltration or hydroxyapatite chromatography (see, e.g., U.S. Pat. No. 5,691,453, which is incorporated herein by reference). Examples of commercially available 100 kD ultrafiltration membranes suitable for performing polymerized hemoglobin fractionation include Pall's 100 kD Omega polyethersulfone (Cassette #, Amersham's polyethersulfone Kvick Flow Process Scale (Cassette # UFEFL0100250 ST) and Millipore's PLCHK composite regenerated cellulose (Cassette # P2C100C25).

Following the pH adjustment, at least one reducing agent, preferably a sodium borohydride solution, is added to the poly(Hb) solution. Typically, about 5 to about 18 moles of reducing agent are added per mole of Hb tetramer (per 64,000 Daltons of Hb) within the poly(Hb).

The pH and electrolytes of the stable poly(Hb) can then be restored to physiologic levels to form a stable polymerized hemoglobin solution, by diafiltering the stable poly(Hb) with a diafiltration solution having a suitable pH and physiologic electrolyte levels.

Wherein the poly(Hb) was reduced by a reducing agent, the diafiltration solution has an acidic pH, preferably between about 4 to about 6.

A non-toxic sulfhydryl compound can also be added to the stable poly(Hb) solution as an oxygen scavenger to enhance the stability of the final polymerized hemoglobin blood-substitute. The sulfhydryl compound can be added as part of the diafiltration solution and/or can be added separately. An amount of sulfhydryl compound is added to establish a sulfhydryl concentration which will scavenge oxygen to maintain methemoglobin content less than about 15% over the storage period. Preferably, the sulfhydryl compound is NAC. Typically, the amount of sulfhydryl compound added is an amount sufficient to establish a sulfhydryl concentration between about 0.05% and about 0.2% by weight.

The polymerized Hb solutions are generally packaged under aseptic handling conditions while maintaining pressure with an inert, substantially oxygen-free atmosphere, in the polymerization reactor and remaining transport apparatus. Such polymerized Hb solutions can then be used for preparing oxygenated Hb solutions of the invention by the methods described above, for example, by the use of oxygenation system 10.

The specifications for a suitable, stable polymerized hemoglobin solution for preparing the oxygenated hemoglobin solutions of the invention are provided in Table I.

TABLE 1

| PARAMETER | RESULTS |
|---|---|
| pH (18-22° C.) | Physiologically acceptable |
| Endotoxin | Physiologically acceptable |
| Sterility Test | Meets Test |
| Phospholipids^{a} | Physiologically acceptable |
| Total Hemoglobin | 10-250 g/l |
| Methemoglobin^{b} | <15% |
| Oxyhemoglobin^{b} | <10% |
| Sodium, Na⁺ | Physiologically acceptable |
| Potassium, K⁺ | |
| Chloride, Cl⁻ | |
| Calcium, Ca⁺⁺ | |
| Boron | |
| Glutaraldehyde | Physiologically acceptable |
| N-acetyl-L-cysteine | Physiologically Acceptable |
| M.W. >500,000 | #18% |
| M.W. <32,000 | <5% |
| Particulate Content >10μ | <12/ml |
| Particulate Content >25μ | <2/ml |

^{a}measured in Hb before polymerization.
^{b}based on the total hemoglobin.

### Exemplification

### EXAMPLE 1

### Oxygenation of Polymerized Hemoglobin Solutions

HEMOPURE® was oxygenated by a method as described above, using oxygenation system 10. In this example, for each 1000 mL product collection bag 16, one 250 mL saline supply bag 18 and one Hb supply bag 12 containing HEMOPURE® were used.

Specific components used for oxygenation system 10 for this example are summarized in Table 2 below:

TABLE 2
System Equipment

| Description | Specifications |
|---|---|
| Minntech Fiberflo Hollow | 0.03 μm pore size, 1 ft² membrane area |
| Fiber Capsule (SV-C-030-P) | |
| for cartridge 20 | |
| Watson-Marlow 323E/D | 0−>100 mL/min flow range, PLC |
| peristaltic pump for pump 46 | controllable |
| Bioprene autoclavable tubing | 4.8 OD × 1.6 mm ID |
| for tubing 17 | |
| Tygon tubing for tubings 11 | 5/32 ID × 7/32 OD |
| and 13 | |
| Male and female Luer | Polypropylene |
| Lock ×1/16 hose barb | |
| for connectors 21 and 23 | |
| Cole Parmer rotameter | 0-60 cc/min range |
| (PMR1-011487) for | |
| rotameter 22 | |
| Watts Fluidair Pressure | 0-300 psig inlet pressure, 0-60 psig |
| Regulator (R364-01AG) for | adjustable outlet pressure |
| pressure regulator 24 | |
| CharterBio BP100BSB bag | Volume 1000 mL |
| assembly w/Needle-less | |
| injection site for connectors | |
| 40 and 42 | |
| Pall 32 mm sterilizing filter | Supor 0.2 μm membrane, Female Luer |
| for filter 38 | Lock inlet, Male Luer Lock outlet |

A medical grade oxygen gas was used and the oxygen gas concentration of oxygen source 14 was greater than 99%. Pressurized oxygen supply was regulated to less than 100 psi, and pressure regulator 24 was rated to a 100 psig inlet pressure. The polymerized hemoglobin solution flow rate was 10-12 mL/min. The oxygen gas flow rate was 10-20 cc/min. Resulting product collection bag 16 in this example contained an oxygenated hemoglobin solution in which a hemoglobin concentration was approximately 6.5±1.0 g/dL and an oxyhemoglobin content was greater than approximately 90%, as summarized in Table 3 below.

TABLE 3
Specifications of Oxygenated HEMOPURE ®

| | Specifications (wt %) |
|---|---|
| Oxy Hemoglobin^{a} | >90% |
| Met Hemoglobin^{a} | <5% |
| total Hemoglobin | 6.5 ± 1.0-12 ± 1.5 g/dL |

^{a}based on the total hemoglobin.

### EXAMPLE 2

### Visualization of Coronary Arteries During OCT Imaging with Oxygenated Polymerized Hemoglobin Solutions

In vitro, attenuation experiments were performed using an OCT microscope (LightLab Imaging) and a tilted diffuse reflector (Spectralon plastic) as a depth reference in H₂O, D₂O and 8 g/dl of an oxygenated HEMOPURE® solution prepared by the method of Example 1. Coronary segments of pigs were imaged with and without stents, and ST depression was followed during up to 2 min occlusion with 0.5 ml/s flushing of saline or the oxygenated HEMOPURE® solution.

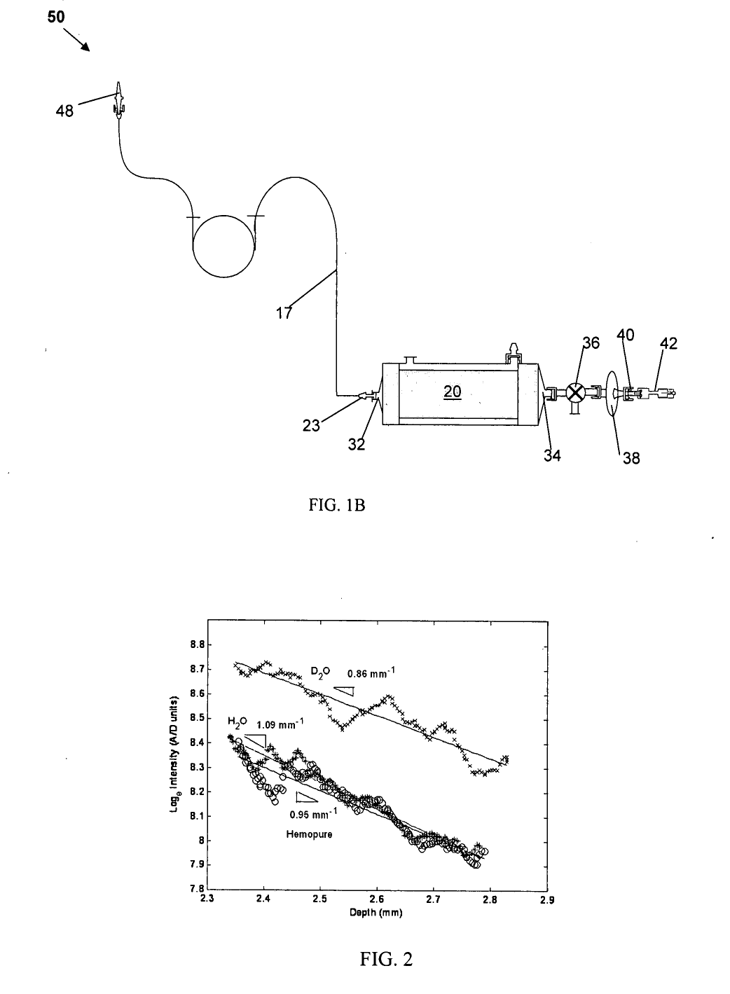

As shown in FIG. 2, attenuation for the oxygenated HEMOPURE® solution (∘) was similar to that for saline (*, referenced as H₂O in FIG. 2) with less than 0.15 mm⁻¹ at 1300 nm. Refractive index of D₂O (x) was 1.357. Clear visualization of coronary wall and stent were possible without ischemia related events with the oxygenated HEMOPURE® solution, whereas 2 min flushing of saline induced ventricular fibrillation. This result indicates that the use of an oxygenated HEMOPURE® solution can provide a safe alternative to saline in OCT imaging.

Equivalents

While this invention has been particularly shown and described with references to preferred embodiments thereof, it will be understood by those skilled in the art that various changes in form and details may be made therein without departing from the scope of the invention encompassed by the appended claims.

What is claimed is:

1. An oxygenated hemoglobin solution, comprising from about 10 grams to about 250 grams of polymerized hemoglobin per liter of solution, wherein:
a) about 80% by weight, or greater, of the polymerized hemoglobin is oxyhemoglobin;
b) about 18% by weight, or less, of the polymerized hemoglobin has a molecular weight of over 500,000 Daltons;
c) about 5% by weight, or less, of the polymerized hemoglobin has a molecular weight equal to or less than 65,000 Daltons;
d) a P₅₀ of the polymerized hemoglobin is in a range of between about 34 and about 46 mm Hg; and
e) an endotoxin content of the hemoglobin solution is less than about 0.05 endotoxin units per milliliter.

2. The oxygenated hemoglobin solution of claim 1, wherein about 90% by weight, or greater, of the polymerized hemoglobin is oxyhemoglobin.

3. The oxygenated hemoglobin solution of claim 1, wherein the polymerized hemoglobin includes bovine-derived hemoglobin.

4. The oxygenated hemoglobin solution of claim 1, wherein the polymerized hemoglobin includes hemoglobin polymerized by a dialdehyde.

5. The oxygenated hemoglobin solution of claim 4, wherein the dialdehyde includes glutaraldehyde.

6. The oxygenated hemoglobin solution of claim 1, wherein the oxyhemoglobin includes ¹⁷O-labeled oxyhemoglobin.

7. The oxygenated hemoglobin solution of claim 1, wherein the oxyhemoglobin includes ¹⁶O-labeled oxyhemoglobin.

8. The oxygenated hemoglobin solution of claim 1, wherein about 15% by weight, or less, of the polymerized hemoglobin is methemoglobin.

9. The oxygenated hemoglobin solution of claim 8, wherein about 5% by weight, or less, of the polymerized hemoglobin is methemoglobin.

10. The oxygenated hemoglobin solution of claim 1, wherein the oxygenated hemoglobin solution includes from about 10 grams to about 100 grams of polymerized hemoglobin per liter of solution.

11. A method of visualizing a tissue, blood vessel or organ of a subject, comprising the steps of:
a) administering to the subject an oxygenated hemoglobin solution, the oxygenated hemoglobin solution including from about 10 grams to about 250 grams of polymerized hemoglobin per liter of solution, wherein:
i) about 80% by weight, or greater, of the polymerized hemoglobin is oxyhemoglobin;
ii) about 18% by weight, or less, of the polymerized hemoglobin has a molecular weight of over 500,000 Daltons;
iii) about 5% by weight, or less, of the polymerized hemoglobin has a molecular weight equal to or less than 65,000 Daltons;
iv) a P₅₀ of the polymerized hemoglobin is in a range of between about 34 and about 46 mm Hg; and
v) an endotoxin content of the hemoglobin solution is less than about 0.05 endotoxin units per milliliter; and
b) imaging the tissue, blood vessel or organ with an imaging system.

12. The method of claim 11, wherein the imaging system is selected from the group consisting of an optical coherence tomography and a magnetic resonance imaging system.

13. The method of claim 12, wherein the tissue or organ is imaged by an optical coherence tomography.

14. The method of claim 13, wherein the oxyhemoglobin includes ¹⁶O-labeled oxyhemoglobin.

15. The method of claim 11, wherein the tissue or organ is imaged by a magnetic resonance imaging system.

16. The method of claim 15, wherein the oxyhemoglobin includes ¹⁷O-labeled oxyhemoglobin.

17. The method of claim 11, wherein the tissue or organ includes a coronary artery, brain, heart, visceral tissue and a transplant.

18. The method of claim 11, wherein about 90% by weight, or greater, of the polymerized hemoglobin is oxyhemoglobin.

19. The method of claim 11, wherein the polymerized hemoglobin includes bovine-derived hemoglobin.

20. The method of claim 11, wherein polymerized hemoglobin includes hemoglobin polymerized by a dialdehyde.

21. The method of claim 20, wherein the dialdehyde includes glutaraldehyde.

22. The method of claim 1, wherein the oxygenated hemoglobin solution is administered to the subject by infusion into the subject's blood stream.

23. A method of preparing an oxygenated hemoglobin solution, comprising the step of oxygenating a hemoglobin solution that includes polymerized hemoglobin using a filter in a single pass-through to thereby cause about 80% by weight, or greater, of the polymerized hemoglobin to become oxyhemoglobin, and wherein:
i) about 18% by weight, or less, of the polymerized hemoglobin has a molecular weight of over 500,000 Daltons;
ii) about 5% by weight, or less, of the polymerized hemoglobin has a molecular weight equal to or less than 65,000 Daltons;
iii) a P₅₀ of the polymerized hemoglobin is in a range of between about 34 and about 46 mm Hg; and
iv) an endotoxin content of the hemoglobin solution is less than about 0.05 endotoxin units per milliliter,
thereby preparing the oxygenated hemoglobin solution.

24. The method of claim 23, wherein the filter is a hydrophobic hollow fiber cartridge where an oxygen gas diffuses into the hemoglobin solution therein and binds the polymerized hemoglobin of the hemoglobin solution to produce oxyhemoglobin.

25. The method of claim 24, wherein the oxygen gas includes an ¹⁷O-labeled oxygen gas.

26. The method of claim 24, wherein the oxygen gas includes an ¹⁶O-labeled oxygen gas.

27. The method of claim 24, wherein the hemoglobin solution to be oxygenated flows through the hydrophobic hollow fiber cartridge at an area normalized flow rate in a range of between about 20 mL/min/m² and about 110 mL/min/m².

28. The method of claim 27, wherein the oxygen gas flows through the hydrophobic hollow fiber cartridge at an area normalized flow rate in a range of between about 50 cc/min/m² and about 300 cc/min/m².

29. The method of claim 28, wherein the hemoglobin solution to be oxygenated flows through the hydrophobic hollow fiber cartridge in a different direction than a direction of flow of the oxygen gas.

30. The method of claim 23, further including the step of adjusting the concentration of the hemoglobin for the oxygenated hemoglobin solution to have from about 10 grams to about 250 grams of polymerized hemoglobin per liter of solution.
